(19) United States
(12) Patent Application Publication
(10) Pub. No.: US 2012/0172457 A1
(43) Pub. Date: Jul. 5, 2012

(54) NOVEL POWDERED POLYMER, PREPARATION METHOD THEREOF AND USE OF SAME AS A THICKENER

(75) Inventors: Olivier Braun, Castres (FR); Paul Mallo, Croissy-Sur-Seine (FR)

(73) Assignee: SOCIETE D'EXPLOITATION DE PRODUITS POUR LES INDUSTRIES CHIMIQUES SEEPIC, Paris (FR)

(21) Appl. No.: 13/395,591

(22) PCT Filed: Sep. 1, 2010
(86) PCT No.: PCT/FR2010/051819

§ 371 Date: Mar. 12, 2012

(30) Foreign Application Priority Data
Sep. 11, 2009 (FR) 0956224

(51) Int. Cl. A61K 47/32; A61K 8/81; A61P 17/00; C08F 236/22

(52) U.S. Cl. 514/772.4; 526/287

(57) ABSTRACT

Disclosed is a branched or crosslinked anionic polyelectrolyte of at least one monomer having a partially- or totally-salified strong acid function, with at least one neutral monomer and at least one monomer having formula (I), wherein: R represents a linear or branched alkyl radical including between 8 and twenty carbon atoms, and n represents a number greater than or equal to 1 and less than or equal to twenty. Also described is the use thereof as a thickener in topical compositions.

The invention relates to novel thickeners as well as use thereof in cosmetics and in pharmacy.

The cosmetics industry and the pharmaceutical industry very regularly use synthetic polymer thickeners for increasing the viscosity of creams, emulsions and various topical solutions. The synthetic polymer thickeners currently used in these fields are in two physical forms, the powder form and the liquid form for which the polymer is prepared by inverse emulsion polymerization by means of surfactants, and which is commonly called inverse latex.

The best-known of the polymer thickeners in powder form are polymers based on acrylic acid or copolymers based on acrylic acid and acrylic acid esters. We may mention for example the polymers marketed by the company Noveon under the brand names CARBOPOL™ and PEMULEN™. They are notably described in the American patents U.S. Pat. No. 5,373,044, U.S. Pat. No. 2,798,053 and in European patent EP 0 301 532. In the cosmetics industry, homopolymers or copolymers based on 2-acrylamido-2-methyl-propanesulfonic acid are also used, again in powder form. These polymer thickeners are marketed under the brand name Aristoflex™ and are notably described in European patents EP 816 403, EP 1 116 733 and EP 1 069 142. These thickeners in powder form are obtained by precipitation polymerization; the monomer(s) is (are) dissolved in an organic solvent such as benzene, ethyl acetate, cyclohexane, tert-butanol. Thickeners in the form of inverse latices and notably those marketed by the applicant are also very widely used in the cosmetics industry. We may mention for example the thickeners Sepigel™ 305, Simulgel™ 600, Simulgel™ EG, Simulgel™ EPG, Simulgel™ NS, Simulgel™A, Sepiplus™ 400, Sepiplus™ 250 and Sepiplus™ 265. These thickeners are obtained by inverse emulsion polymerization. They have the advantage of easier handling, and they disperse in water very quickly. Moreover, these products develop remarkably high thickening performance; this performance probably results from their method of preparation, a dispersed-phase polymerization reaction, which leads to polymers with very high molecular weights.

The international application published under number WO 2006/002936 discloses water-soluble copolymers, which are used as water retention agents, stabilizers and rheology modifiers for cements, plasters, mortars, water-based paints and other compositions based on aqueous binders. These copolymers are obtained from monomers with a sulfonic acid function, monomers with an amine function and monomers derived from polyethoxylated alkyl (meth)acrylate. This international application discloses, more particularly, a terpolymer of partially salified or completely salified 2-methyl 2-[(1-oxo 2-propenyl) amino] 1-propanesulfonic acid, of acrylamide and of pentacosa-ethoxylated stearyl methacrylate (monomer marketed under the name PLEX 6877-O™.

However, these thickeners in the form of inverse latex contain an oil and one or more surfactants, which can sometimes induce reactions of cutaneous intolerance in subjects who are particularly sensitive; moreover, said presence of oil makes them unusable for preparing clear gels.

The inventors have therefore developed thickener systems like those described in the French patent application published under number 2 910 899, which discloses a linear, branched or crosslinked terpolymer of at least one monomer possessing a strong acid function, free, partially salified or completely salified, with at least one neutral monomer, and at least one monomer of formula (A):

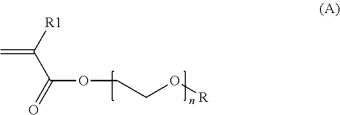

In (A), R1 represents a hydrogen atom or a methyl radical, R represents a linear or branched alkyl radical having from eight to thirty carbon atoms and n represents a number greater than or equal to one and less than or equal to fifty. These polymers display very pronounced thickening properties, notably in the presence of electrolytes. They function over a wide range of pH and can be used for making transparent gels. However, the low-pH formulations thickened with certain of them do not have satisfactory long-term resistance to salts and some of them, which contain fatty alcohols, have a rather unappealing elastic appearance and produce sensations of stickiness to the touch and/or have the appearance of a cream or an emulsion that is grainy and discontinuous.

The applicant demonstrated that these drawbacks could be avoided by selecting certain of these terpolymers that were not disclosed in the French patent application published under number 2 910 899.

That is why according to a first aspect the invention relates to a branched or crosslinked anionic polyelectrolyte resulting from the polymerization of partially salified or completely salified 2-methyl 2-[(1-oxo 2-propenyl) amino] 1-propanesulfonic acid, with at least one neutral monomer selected from acrylamide, (2-hydroxy-ethyl) acrylate or N,N-dimethyl acrylamide, and at least one monomer of formula (I):

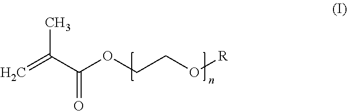

in which R represents a linear or branched alkyl radical having from eight to twenty carbon atoms and n represents a number greater than or equal to one and less than or equal to twenty, selected from tetraethoxylated lauryl methacrylate or eicosaethoxylated stearyl methacrylate in the presence of at one crosslinking agent.

“Crosslinked polymer” denotes a nonlinear polymer that is in the form of a three-dimensional network, insoluble in water, but swellable in water and therefore leading to the production of a chemical gel.

According to another particular embodiment, the anionic polyelectrolyte is crosslinked with a diethylene or polyethylene compound in the molar proportions, expressed relative to the monomers employed, from 0.005% to 1%, more particularly from 0.01% to 0.5% and quite particularly from 0.01% to 0.25%.

The crosslinking agent is more particularly selected from ethylene glycol dimethacrylate, tetraallyloxyethane, ethylene glycol diacrylate, diallyl urea, triallyl amine, trimethylol propanetriacrylate or methylene-bis(acrylamide) or a mixture of its compounds.

In the context of the present invention, the anionic polyelectrolyte as defined above generally comprises between 5 mol. % and 95 mol. % of monomers with a strong acid function, more particularly between 10 mol. % and 90 mol. % and quite particularly between 20 mol. % and 80 mol. %.

In the context of the present invention, the strong acid function is partially or completely salified as alkali metal salt such as for example sodium salt or potassium salt or as ammonium salt.

According to another particular aspect, the invention relates to an anionic polyelectrolyte as defined above, in which the monomer with strong acid function is 2-methyl 2-[(1-oxo 2-propenyl) amino] 1-propanesulfonic acid partially or completely salified in the form of ammonium salt.

In the context of the present invention the anionic polyelectrolyte as defined above more particularly comprises between 4.9 mol. % and 90 mol. % of neutral monomer, more particularly between 9.5 mol. % and 85 mol. % and quite particularly between 15 mol. % and 75 mol. %.

According to a more particular embodiment, the invention relates to an anionic polyelectrolyte as defined above, in which the neutral monomer is N,N-dimethyl acrylamide.

According to another more particular embodiment, the invention relates to an anionic polyelectrolyte as defined above, in which the neutral monomer is acrylamide.

According to another more particular embodiment, the invention relates to an anionic polyelectrolyte as defined above, in which the neutral monomer is (2-hydroxy-ethyl) acrylate.

According to a quite particular embodiment, the invention relates to an anionic polyelectrolyte as defined above, in which the monomer of formula (I) is tetraethoxylated lauryl methacrylate, corresponding to the compound of formula (I) in which the radical R represents the dodecyl radical and n is equal to 4.

According to a quite particular embodiment, the invention relates to an anionic polyelectrolyte as defined above, in which the monomer is eicosaethoxylated stearyl methacrylate, corresponding to the compound of formula (I) in which the radical R represents the octadecyl radical and n is equal to 20.

In the context of the present invention, the anionic polyelectrolyte as defined above comprises between 0.1 mol. % and 10 mol. % of monomers of formula (I) and more particularly between 0.5 mol. % and 5 mol. %.

The polyelectrolyte according to the present invention can also comprise various additives, such as complexing agents, chain transfer agents or chain limiting agents.

The invention relates more particularly to an anionic polyelectrolyte selected from the following polymers:
Terpolymer of 2-methyl 2-[(1-oxo 2-propenyl) amino] 1-propanesulfonic acid completely salified in the form of ammonium salt, acrylamide and tetraethoxylated lauryl methacrylate, crosslinked with trimethylol propanetriacrylate;
Terpolymer, of 2-methyl 2-[(1-oxo 2-propenyl) amino] 1-propanesulfonic acid completely salified in the form of ammonium salt, acrylamide and eicosaethoxylated stearyl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and eicosaethoxylated stearyl methacrylate, crosslinked with trimethylol propanetriacrylate;
Terpolymer of 2-methyl 2-[(1-oxo 2-propenyl) amino] 1-propanesulfonic acid completely salified in the form of ammonium, N,N-dimethyl acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, N,N-dimethyl acrylamide and eicosaethoxylated stearyl methacrylate, crosslinked with trimethylol propanetriacrylate.

According to another particular aspect, the invention relates to a crosslinked anionic polyelectrolyte, as defined above, comprising for 100% of monomers used:
From 20 mol. % to 80 mol. % of monomer units derived from a monomer having a strong acid function;
From 15 mol. % to 75 mol. % of monomer units derived from a neutral monomer;
From 0.5% to 5 mol. % of monomer units derived from a monomer of formula (I) as defined above.

According to another more particular aspect, the invention relates to a crosslinked anionic polyelectrolyte, as defined above, comprising for 100% of monomers used:
From 55 mol. % to 80 mol. % of monomer units derived from a monomer having a strong acid function;
From 15 mol. % to 40 mol. % of monomer units derived from a neutral monomer;
From 1% to 5 mol. % of monomer units derived from a monomer of formula (I) as defined above.

The invention also relates to a method of preparing the polyelectrolyte as defined above, characterized in that:
a) all of the monomers, the crosslinking agent and/or the other additives are mixed in an organic solvent, preferably tert-butanol,
b) the polymerization reaction is initiated by introducing a free radical initiator into the dispersion prepared in step a), and it is then left to develop,
c) when polymerization has ended, the precipitate is collected by evaporation of the solvent or filtration, then
d) the precipitate is dried.

According to a preferred implementation of the method as defined above, the polymerization reaction is initiated at a temperature greater than or equal to 50° C. by means of a radical initiator producing radicals by homolysis, such as dilauroyl peroxide, azo-bis-isobutyronitrile or azo derivatives.

According to another implementation of the method as defined above, the polymerization reaction is initiated by a redox pair, at a temperature less than or equal to 20° C.

The invention also relates to the use of the anionic polyelectrolyte as defined above, as thickener and/or as stabilizer and/or as emulsifier, of a cosmetic, dermopharmaceutical or pharmaceutical topical composition.

A topical composition according to the invention, intended to be applied on the skin or the mucosae of humans or animals, can comprise a topical emulsion comprising at least one aqueous phase and at least one oily phase. This topical emulsion can be of the oil-in-water (O/W), water-in-oil (W/O), oil-in-water-in-oil (O/W/O) or water-in-oil-in-water (W/O/W) type. The oily phase of the topical emulsion can comprise a mixture of one or more oils.

A topical composition according to the invention can be intended for cosmetic use or to be used for preparing a medicinal product intended for treating diseases of the skin, scalp and mucosae. In the last-mentioned case, the topical composition then comprises an active principle which can for example comprise an anti-inflammatory agent, a muscle relaxant, an antifungal, an antibacterial or an antidandruff agent.

When the topical composition is used as a cosmetic composition intended to be applied on the skin, scalp or mucosae, it may or may not comprise an active principle, for example a hydrating agent, a tanning agent, a sun filter, an antiwrinkle, an agent for the purpose of slimming, an antiradical agent, an antiacne agent, an antifungal or antidandruff agent.

The invention finally relates to a topical composition according to the invention usually comprises between 0.1% and 10 wt. % and more particularly from 1 to 5 wt. % of the anionic polyelectrolyte as defined above. The pH of the topical composition is preferably greater than or equal to 3.

The topical composition can further comprise compounds conventionally included in compositions of this type, for example perfumes, preservatives, dyes, pigments, sunscreens, active ingredients, emollients or surfactants.

According to another particular aspect, the invention relates to the use of the anionic polyelectrolyte as defined above, for thickening and emulsifying and stabilizing a topical composition comprising at least one aqueous phase.

The anionic polyelectrolyte according to the invention is an interesting substitute for the inverse latices sold under the names SEPIGEL™ 305, SEPIGEL™ 501, SIMULGEL™ EG, SIMULGEL™ EPG, SIMULGEL™ NS, SIMULGEL™ 600, SIMULGEL™ A, SEPIPLUS™ 265, SEPIPLUS™ 250, SEPIPLUS™ 400 or SEPINOV™ EMT 10 by the applicant, as it also displays good compatibility with the other excipients used for preparing formulations such as milks, lotions, creams, soaps, baths, balsams, shampoos or conditioners. It can also be used with said SEPIGEL™ or SIMULGEL™, SEPIPLUS™ and/or SEPINOV™ EMT 10.

Notably it is compatible with the concentrates described and claimed in international publications WO 92/06778, WO 95/04592, WO 95/13863, WO 96/37285, WO 98/22207, WO 98/47610 or in FR 2 734 496, with the surfactants described in WO 93/08204. It is particularly compatible with MONTANOV™ 68, MONTANOV™ 82, MONTANOV™ 202, MONTANOV™ L, MONTANOV™ S, FLUIDANOV™ 20X or EASYNOV™. It can also be used in emulsions of the type such as those described and claimed in EP 0 629 396 and in cosmetically or physiologically acceptable aqueous dispersions with an organopolysiloxane compound selected for example from those described in WO 93/05762 or in WO 93/21316.

It can also be used to form cosmetically or physiologically acceptable aqueous gels with acid pH, such as those described in WO 93/07856; it can also be used together with nonionic celluloses, for example to form styling gels such as those described in EP 0 684 024, or together with esters of fatty acids and of sugar, to form compositions for treating the hair or the skin such as those described in EP 0 603 019, or in shampoos or conditioners as described and claimed in WO 92/21316 or finally together with an anionic homopolymer such as CARBOPOL™ to form hair treatment products such as those described in DE 19523596.

It is also compatible with the N-acylated derivatives of amino acids, which means it can be used in soothing compositions notably for sensitive skin, such as those described or claimed in WO 92/21318, WO 94/27561 or WO 98/09611. It is also compatible with thickening and/or gelling polymers such as hydrocolloids of vegetable origin or biosynthetic, for example xanthan gum, karaya gum, carrageenates, alginates, galactomannans; such as silicates; such as cellulose and derivatives thereof; such as starch and hydrophilic derivatives thereof; such as polyurethanes.

A) Preparation of Polyelectrolytes:

### EXAMPLE 1

### (According to the Invention)

### Terpolymer of ammonium 2-methyl 2-[(1-oxo 2-propenyl) amino] 1-propanesulfonate, N,N-dimethyl acrylamide and tetraethoxylated lauryl methacrylate [AMPS/DMAM/MAL(4OE) 77.4/19.2/3.4 molar], crosslinked with trimethylol propanetriacrylate (TMPTA)

A reactor maintained at 25° C. with stirring is loaded with 592 g of a 15 wt. % aqueous solution of ammonium 2-methyl 2-[(1-oxo 2-propenyl) amino] 1-propanesulfonate in a tert-butanol/water mixture (97.5/2.5 by volume), 10.1 g of N,N-dimethyl acrylamide, 4.2 g of tetraethoxylated lauryl methacrylate and 0.75 g of trimethylol propanetriacrylate. After a sufficient time to achieve good homogenization of the solution, the latter is deoxygenated by bubbling with nitrogen heated to 70° C. Then 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte 1.

### EXAMPLE 2

### (According to the Invention)

### Terpolymer of ammonium 2-methyl 2-[(1-oxo 2-propenyl) amino] 1-propanesulfonate, N,N-dimethyl acrylamide and eicosaethoxylated stearyl methacrylate [AMPS/DMAM/MAS(20OE) 79.3/19.7/1 molar], crosslinked with trimethylol propanetriacrylate (TMPTA)

A reactor maintained at 25° C. with stirring is loaded with 592g of a 15 wt. % aqueous solution of ammonium 2-methyl 2-[(1-oxo 2-propenyl) amino] 1-propanesulfonate in a tert-butanol/water mixture (97.5/2.5 by volume), 10.1 g of N,N-dimethyl acrylamide, 6.2 g of eicosaethoxylated stearyl methacrylate and 0.75 g of trimethylol propanetriacrylate. After a sufficient time to achieve good homogenization of the solution, the latter is deoxygenated by bubbling with nitrogen heated to 70° C. Then 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte 2.

### EXAMPLE 3

### (According to the Invention)

### Terpolymer of ammonium 2-methyl 2-[(1-oxo 2-propenyl) amino] 1-propanesulfonate, 2-hydroxy-ethyl acrylate and tetraethoxylated lauryl methacrylate, [(AMPS/HEA/MAL(4OE) 68.7/29.5/1.8 molar], crosslinked with trimethylol propanetriacrylate (TMPTA)

A reactor maintained at 25° C., with stirring, is loaded with 592 g of a 14% solution of the ammonium salt of 2-methyl 2-[(1-oxo 2-propenyl) amino] 1-propanesulfonic acid in a tert-butanol/water mixture (97.5/2.5 by volume), 18.5 g of hydroxy-ethyl acrylate, 4.2 g of tetraethoxylated lauryl methacrylate and 0.75 g of trimethylol propanetriacrylate. After a sufficient time to achieve good homogenization of the solution, the latter is deoxygenated by bubbling with nitrogen heated to 70° C. Then 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte 3.

### EXAMPLE 4

### (According to the Invention)

### Terpolymer of ammonium 2-methyl 2-[(1-oxo 2-propenyl) amino] 1-propanesulfonate, acrylamide and tetraethoxylated lauryl methacrylate, [AMPS/AM/MAL(4OE) 61.2/37.2/1.6 molar] crosslinked with trimethylol propanetriacrylate (TMPTA)

A reactor maintained at 25° C. with stirring is loaded with 592g of a 14% solution of the ammonium salt of 2-methyl 2-[(1-oxo 2-propenyl) amino] 1-propanesulfonic acid in a tert-butanol/water mixture (97.5/2.5 by volume), 16 g of acrylamide crystal, 4.2 g of tetraethoxylated lauryl methacrylate and 0.75 g of trimethylol propanetriacrylate. After a sufficient time to achieve good homogenization of the solution, the latter is deoxygenated by bubbling with nitrogen heated to 70° C. Then 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte 4

### EXAMPLE T1

### (According to the Prior Art)

### Terpolymer of ammonium 2-methyl 2-[(1-oxo 2-propenyl) amino] 1-propanesulfonate, N,N-dimethyl acrylamide and tetraethoxylated lauryl acrylate, [AMPS/DMAM/AL(4OE), 77.4/19.2/3.4] crosslinked with trimethylol propanetriacrylate (TMPTA)

A reactor maintained at 25° C., with stirring, is loaded with 592 g of a 15.5% solution of the ammonium salt of 2-methyl 2-[(1-oxo 2-propenyl) amino] 1-propanesulfonic acid in a tert-butanol/water mixture (97.5/2.5 by volume), 10.1 g of N,N-dimethyl acrylamide, 4.1 g of tetraethoxylated lauryl acrylate and 0.75 g of trimethylol propanetriacrylate.

After a sufficient time to achieve good homogenization of the solution, it is deoxygenated by bubbling with nitrogen and then the temperature of the reaction mixture is raised to 70° C. When the desired temperature has been reached, 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte T1.

### Example T2

### (According to the Prior Art)

### Terpolymer of ammonium 2-methyl 2-[(1-oxo 2-propenyl) amino] 1-propanesulfonate, N,N-dimethyl acrylamide and eicosaethoxylated stearyl acrylate [AMPS/DMAM/AS(20OE), 79.3/19.7/1 molar] crosslinked with trimethylol propanetriacrylate

A reactor maintained at 25° C., with stirring, is loaded with 592 g of a 15.5% solution of the ammonium salt of 2-methyl 2-[(1-oxo 2-propenyl) amino] 1-propanesulfonic acid in a tert-butanol/water mixture (97.5/2.5 by volume), 10.1 g of N,N-dimethyl acrylamide, 6.1 g of eicosaethoxylated stearyl methacrylate and 0.5 g of trimethylol propanetriacrylate. After a sufficient time to achieve good homogenization of the solution, the latter is deoxygenated by bubbling with nitrogen heated to 70° C. Then 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte T2

### EXAMPLE T3

### (According to the Prior Art)

### Terpolymer of ammonium 2-methyl 2-[(1-oxo 2-propenyl) amino] 1-propanesulfonate, N,N-dimethyl acrylamide and pentacosaethoxylated behenyl methacrylate [AMPS/DMAM/MAB(25OE), 79.3/19.7/1], crosslinked with trimethylol propanetriacrylate;

A reactor maintained at 25° C., with stirring, is loaded with 592 g of a 15.5% solution of the ammonium salt of 2-methyl 2-[(1-oxo 2-propenyl) amino] 1-propanesulfonic acid in a tert-butanol/water mixture (97.5/2.5 by volume), 10.1 g of N,N-dimethyl acrylamide, 7.7 g of pentacosaethoxylated behenyl methacrylate, (1500=MW) and 0.75 g of trimethylol propanetriacrylate. After a sufficient time to achieve good homogenization of the solution, the latter is deoxygenated by bubbling with nitrogen heated to 70° C. Then 0.42 g of dilauroyl peroxide is added and the reaction mixture is then held at 70° C. for about 60 minutes and then at 80° C. for 2 hours. After cooling, the powder that formed during polymerization is filtered and dried, obtaining the desired product, called hereinafter: Polyelectrolyte T3.

B) Comparative Investigations:

The thickeners according to the invention and those according to the prior art were evaluated as follows:

a) Measurement of viscosities (η):
at 2 wt. % in water,
at 2 wt. % in water at pH 3.5
and at 2 wt. % in water at pH 3.5 and comprising 2% of sodium chloride.

These measurements of viscosity, expressed in mPas, were carried out at 25° C. using a type RVT Brookfield rheometer equipped with spindle number 6 and set to a rotary speed of 5 revolutions per minute (rpm). The pH was adjusted to 3.5 by adding glycolic acid and soda if necessary. The measurements were performed just after preparation T=0 at T=1 month (M1) then at T=3 months (M3).

b) Application formulas for assessment were prepared, thickened with each of the polyelectrolytes 1, 2, 3, 4, T1, T2 and T3 of the following composition:
Glycolic acid: 4 wt. %
Thickener: 2 wt. %
Isohexadecane: 15 wt. %
Mixture of cetyl alcohol and behenyl alcohol: 2 wt. %
SEPICIDE™ HB: 0.5 wt. %
NaOH: qs pH 3.5
Water: q.s.

These formulas are assessed in terms of visual appearance, sensory properties and stability of the formula after 24 hours. The properties found for the polyelectrolytes and the compositions according to the invention are presented in the following table, where they are compared with polyelectrolytes and compositions according to the prior art.

| Polyelectrolyte --> | 1 | T1 | 2 | T2 |
|---|---|---|---|---|
| η at 2% in H₂O | 45 000 | 48 000 | 80 000 | 100 000 |
| η at 2% in H₂O pH = | 53 000 | 50 000 | 65 000 | 52 000 |
| 3.5 | | | | |
| η at 2% in H₂O + 2% | 45 000 | 45 000 | 60 000 | 45 000 |
| NaCl | | | | |
| pH = 3.5 at T0 | | | | |
| η at 2% in H₂O + 2% | 44 000 | 40 000 | 60 000 | 40 000 |
| NaCl | | | | |
| pH = 3.5 at M1 | | | | |
| η at 2% in H₂O + 2% | 45 000 s | 30 000 | 57 000 | 35 000 |
| NaCl | | | | |
| pH = 3.5 at M3 | | | | |
| Appearance of | Smooth | Rubbery | Smooth | Smooth and |
| assessment formula | and | | and | continuous |
| | continuous | | continuous | |
| Sensory property | Pleasant | Slightly | Pleasant | Pleasant |
| | and soft | sticky | and soft | and soft |
| Stability of the | η stable | Large | η stable | Slight |
| formula after 24 H | | increase in | | increase in |
| | | η | | η |

| Polyelectrolyte --> | 3 | 4 | T3 |
|---|---|---|---|
| η at 2% in H₂O | 50 000 | 75 000 | 180 000 |
| η at 2% in H₂O pH = 3.5 | 55 000 | 70 000 | 50 000 |
| η at 2% in H₂O + 2% NaCl pH = 3.5 | 52 000 | 60 000 | 32 400 |
| at T0 | | | |
| η at 2% in H₂O + 2% NaCl pH 3.5 at | 49 000 | 60 000 | 32 000 |
| M1 | | | |
| η at 2% in H₂O + 2% NaCl pH 3.5 at | 47 000 | 57 000 | 28 000 |
| M3 | | | |
| Appearance of assessment formula | Smooth | Smooth | Rubbery and |
| | and | and | grainy |
| | continuous | continuous | |
| Sensory property | Pleasant | Pleasant | Sticky |
| | and soft | and soft | |
| Stability of the formula after | η stable | η stable | Large |
| 24 H | | | increase in |
| | | | η |

These results demonstrate that the polyelectrolytes according to the present invention give compositions that are more resistant to salts, owing to the presence of the monomer of formula (I) within them.

C) Examples of Formulations Prepared with Polyelectrolytes 1, 2, 3 or 4 According to the Invention

### EXAMPLE 5

### Care Cream (Proportions Expressed in Percentages by Weight)

Cyclomethicone: 10%
Polyelectrolyte 1: 0.8%
MONTANOV™ 68: 2%
Stearyl alcohol: 1%
Stearic alcohol: 0.5%
Preservative: 0.65%
Lysine: 0.025%
EDTA (disodium salt): 0.05%
Xanthan gum: 0.2%
Glycerol: 3%
Water: q.s.f. 100%

### EXAMPLE 6

### After-Shave Balsam

Formula
A Polyelectrolyte 2: 1.5%
Water: q.s.f. 100%
B MICROPEARL™ M 100: 5.0%
SEPICIDE™ CI: 0.50%
Perfume: 0.20%
Ethanol 95°: 10.0%

Procedure

Add B to A.

### EXAMPLE 7

### Satiny Emulsion for the Body

Formula
A SIMULSOL™ 165: 5.0%
LANOL™ 1688: 8.50%
Shea butter: 2%
Paraffin oil: 6.5%
LANOL™ 14M: 3%
LANOL™ S: 0.6%
B Water: 66.2%
C MICROPEARL™ M 100: 5%
D Polyelectrolyte 3: 3%
E SEPICIDE™ CI: 0.3%
SEPICIDE™ HB: 0.5%
AQUAXYL™: 3%
Perfume: 0.20%
Vitamin E acetate: 0.20%
Sodium pyrrolidinonecarboxylate: 1%

Procedure

Add C to B, emulsify B in A at 70° C., then add D at 60° C. then E at 30° C.

### EXAMPLE 8

### O/W Cream

Formula
A SIMULSOL™ 165: 5.0%
LANOL™ 1688: 20.0%
LANOL™ P: 1.0%
B Water: q.s.f. 100%
C Polyelectrolyte 4: 2.50%
D SEPICIDE™ CI: 0.20%

SEPICIDE™ HB: 0.30%

Procedure

Add B to A at about 75° C.; add C at about 60° C., then D at about 45° C.

### EXAMPLE 9

### Non-Greasy Sun Gel

Formula
Water: 30%
B SEPICIDE™ CI: 0.20%
SEPICIDE™ HB: 0.30%
Perfume: 0.10%
C Dye: q.s.
Water: 30%
D MICROPEARL™ M 100: 3.00%
Water: q.s.f. 100%
E Silicone oil: 2.0%
PARSOL™ MCX: 5.00%

Procedure

Add B to A; add C, then D, then E.

### EXAMPLE 10

### Sun Filter Milk

Formula
A MONTANOV™ S: 3.0%
Sesame oil: 5.0%
PARSOL™ MCX: 5.0%
Carrageenan 0.10%
B Water: q.s.f. 100%
C Polyelectrolyte 3: 0.80%
D Perfume: q.s.
Preservative: q.s.

Procedure

Emulsify B in A at 75° C. then add C at about 60° C., then D at about 30° C. and adjust the pH if necessary.

### EXAMPLE 11

### Massage Gel

Formula
A Polyelectrolyte 2: 3.5%
Water: 20.0%
B Dye: 2 drops/100 g
Water: q.s.
C Ethanol: 10%
Menthol: 0.10%
D Silicone oil: 5.0%

Procedure

Add B to A, then add to the mixture, C then D

### EXAMPLE 12

### Moisturizing and Matting Foundation

Formula
A Water: 20.0%
Butylene glycol: 4.0%
PEG-400: 4.0%
PECOSIL™ PS100: 1.0%
Sodium hydroxide: q.s. pH=9
Titanium dioxide: 7.0%
Talc: 2.0%
Yellow iron oxide: 0.8%
Red iron oxide: 0.3%
Black iron oxide: 0.05%
B LANOL™ 99: 8%
Caprylic capric triglyceride 8%
MONTANOV™ 202: 5.00%
C Water: q.s.f. 100%
MICROPEARL™ M305: 2.0%
EDTA tetrasodium: 0.05%
D Cyclomethicone: 4.0%
Xanthan gum: 0.2%
Polyelectrolyte 1: 0.8%
E SEPICIDE™ HB: 0.5%
SEPICIDE CI: 0.3%
Perfume: 0.2%

Procedure

Prepare mixtures B+D and A+C at 80° C., then mix and emulsify together.

### EXAMPLE 13

### Glamour Gel

Formula
A Polyelectrolyte 1: 4%
Water: 30%
B ELASTIN HPM: 5.0%
C MICROPEARL™ M 100: 3%
Water: 5%
D SEPICIDE™ CI: 0.2%
SEPICIDE™ HB: 0.3%
Perfume: 0.06%
Sodium pyrrolidinonecarboxylate 50%: 1%
Water: q.s.f. 100%

Procedure

Prepare A; add B, then C, then D.

### EXAMPLE 14

### Body Milk

MONTANOV™ S: 3.5%
LANOL™ 37T: 8.0%
SOLAGUM™ L: 0.05%
Water: q.s.f. 100%
Benzophenone-3: 2.0%
Dimethicone 350 cPs: 0.05%
Polyelectrolyte 3: 0.8%
Preservative: 0.2%
Perfume: 0.4%

### EXAMPLE 15

### Makeup-Removal Emulsion with Sweet Almond Oil

MONTANOV™ 68: 5%
Sweet almond oil: 5%
Water: q.s.f. 100%
Polyelectrolyte 3: 0.3%
Glycerol: 5%
Preservative: 0.2%
Perfume: 0.3%

### EXAMPLE 16

### Moisturizing Cream for Greasy Skin

MONTANOV™ 68: 5%
Cetylstearyloctanoate: 8%
Octyl palmitate: 2%
Water: q.s.f. 100%
Polyelectrolyte 1: 0.6%
MICROPEARL™ M100: 3.0%
Mucopolysaccharides: 5%
SEPICIDE™ HB: 0.8%
Perfume: 0.3%

### EXAMPLE 17

### Soothing After-Shave Balsam, Alcohol-Free

LIPACIDE™ PVB: 1.0%
LANOL™ 99: 2.0%
Sweet almond oil: 0.5%
Polyelectrolyte 1: 3.5%
Water: q.s.f. 100%
Perfume: 0.4%
SEPICIDE™ HB: 0.4%
SEPICIDE™ CI: 0.2%

### EXAMPLE 18

### Cream with AHAs for Sensitive Skin

Mixture of N-lauroyl amino acids: 0.1% to 5%
Magnesium aspartate and potassium aspartate: 0.002% to 0.5%
LANOL™ 99: 2%
MONTANOV™ 68: 5.0%
Water: q.s.f. 100%
Polyelectrolyte 4: 1.50%
Gluconic acid: 1.50%
Triethanolamine (TEA): 0.9%
SEPICIDE™ HB: 0.3%
SEPICIDE™ CI: 0.2%
Perfume: 0.4%

### EXAMPLE 19

### Soothing After-Sun Care

Mixture of N-lauroyl amino acids: 0.1% to 5%
Magnesium aspartate and potassium aspartate: 0.002% to 0.5%
LANOL™ 99: 10.0%
Water: q.s.f. 100%
Polyelectrolyte 1: 2.50%
SEPICIDE™ HB: 0.3%
SEPICIDE™ CI: 0.2%
Perfume: 0.4%
Dye: 0.03%

### EXAMPLE 20

### Milk for Makeup Removal

MONTANOV™ S: 3%
PRIMOL™ 352: 8.0%
Sweet almond oil: 2%
Water: q.s.f. 100%
Polyelectrolyte 2: 0.8%
Preservative: 0.2%

### EXAMPLE 21

### Fluid Emulsion at Alkaline pH

MARCOL™ 82: 5.0%
Sodium hydroxide: 10.0%
Water: q.s.f. 100%
Polyelectrolyte 3: 1.5%

### EXAMPLE 22

### Fluid Foundation

SIMULSOL™ 165: 5.0%
LANOL™ 84D: 8.0%
LANOL™ 99: 5.0%
Water: q.s.f. 100%
Pigments and mineral fillers: 10.0%
Polyelectrolyte 4: 1.2%
Preservative: 0.2%
Perfume: 0.4%

### Example 23

### Sun Filter Milk

MONTANOV™ S: 3.5%
LANOL™ 37T: 10.0%
PARSOL™ MCX: 5.0%
EUSOLEX™ 4360: 2.0%
Water: q.s.f. 100%
Polyelectrolyte 3: 1.8%
Preservative: 0.2%
Perfume: 0.4%

### EXAMPLE 24

### Eye Contour Gel

Polyelectrolyte 2: 2.0%
Perfume: 0.06%
Sodium pyrrolidinonecarboxylate: 0.2%
DOW CORNING™ 245 Fluid: 2.0%
Water: q.s.f. 100%

### EXAMPLE 25

### Non-Rinsed Care Composition

Polyelectrolyte 2: 1.5%
Perfume: q.s
Preservative: q.s.
DOW CORNING™ X2 8360: 5.0%
DOW CORNING™ Q2 1401: 15.0%
Water: q.s.f. 100%

### EXAMPLE 26

### Slimming Gel

Polyelectrolyte 4: 5%
Ethanol: 30%
Menthol: 0.1%
Caffeine: 2.5%
Butcher's broom extract: 2%
Ivy extract: 2%
SEPICIDE™ HB: 1%
Water: q.s.f. 100%

### EXAMPLE 27

### Cream Gel Tinted Ultra Natural

Formula
A Water: 10.0%
Butylene glycol: 4.0%
PEG-400: 4.0%
PECOSIL™ PS100: 1.5%
NaOH: q.s. pH=7
Titanium dioxide: 2.0%
Yellow iron oxide: 0.8%
Red iron oxide: 0.3%
Black iron oxide: 0.05%
B LANOL™ 99: 4.0%
Caprylic capric triglyceride 4.0%
SEPIFEEL™ ONE: 1.0%
Polyelectrolyte 2: 3.0%
C Water: q.s.f. 100%
MICROPEARL™ M305: 2.0%
EDTA tetrasodium: 0.05%
Cyclomethicone: 4.0%
D SEPICIDE™ HB: 0.5%
SEPICIDE CI: 0.3%
Perfume: 0.2%

Procedure

Prepare mixture B+C then add A then D.

### EXAMPLE 28

### Care for Greasy Skin

MICROPEARL™ M310: 1.0%
Polyelectrolyte 3: 5.0%
Octyl isononanoate: 4.0%
Water: q.s.f. 100%
SEPICONTROL™ A5: 4.0%
Perfume: 0.1%
SEPICIDE™ HB: 0.3%
SEPICIDE™ CI: 0.2%
CAPIGEL™ 98: 0.5%
Water: 10%

### EXAMPLE 29

### Cream with AHAs

MONTANOV™ 68: 5.0%
LIPACIDE™ PVB: 1.05%
LANOL™ 99: 10.0%
Water: q.s.f. 100%
Gluconic acid: 1.5%
TEA (triethanolamine): 0.9%
Polyelectrolyte 4: 1.5%
Perfume: 0.4%
SEPICIDE™ HB: 0.2%
SEPICIDE™ CI: 0.4%

### EXAMPLE 30

### Non-Greasy Self-Tanning for Face and Body

LANOL™ 2681: 3.0%
Water: q.s.f. 100%
Dihydroxyacetone: 3.0%
Perfume: 0.2%
SEPICIDE™ HB: 0.8%
Sodium hydroxide: q.s. pH=5

### EXAMPLE 31

### Sun Filter Milk with Tahiti Monoi

Tahiti monoi: 10%
LIPACIDE™ PVB: 0.5%
Polyelectrolyte 1: 2.2%
Water: q.s.f. 100%
Perfume: 0.1%
SEPICIDE™ HB: 0.3%
SEPICIDE™ CI: 0.1%
PARSOL™ MCX: 4.0%

### EXAMPLE 32

### Sun Care for the Face

Cyclomethicone and Dimethiconol: 4.0%
Polyelectrolyte 2: 3.5%
Water: q.s.f. 100%
Perfume: 0.1%
SEPICIDE™ HB: 0.3%
SEPICIDE™ CI: 0.21%
PARSOL™ MCX: 5.0%
Titanium mica: 2.0%
Lactic acid: q.s.f. pH=6.5

### EXAMPLE 33

### Selftanning Emulsion

LANOL™ 99: 15%
MONTANOV™ 68: 5.0%
PARSOL™ MCX: 3.0%
Water: q.s.f. 100%
Dihydroxyacetone: 5.0%
Monosodium phosphate: 0.2%
Polyelectrolyte 3: 0.5%
Perfume: 0.3%
SEPICIDE™ HB: 0.8%
Sodium hydroxide: q.s. pH=5.

### EXAMPLE 34

### Care Cream

Cyclomethicone: 10%
Polyelectrolyte 4: 0.8%
MONTANOV™ 68: 4.5%
Preservative: 0.65%
Lysine: 0.025%
EDTA (disodium salt): 0.05%
Xanthan gum: 0.2%
Glycerol: 3%
Water: q.s. 100%

### EXAMPLE 35

### Care Cream

Cyclomethicone: 10%
Polyelectrolyte 3: 0.8%
MONTANOV™ 68: 4.5%
Perfluoropolymethylisopropyl ether: 0.5%
Preservative: 0.65%
Lysine: 0.025%
EDTA (disodium salt): 0.05%
PEMULEN™ TR1: 0.2%
Glycerol: 3%
Water: q.s. 100%

### EXAMPLE 36

### Body Milk

Formula
A SIMULSOL™ 165: 5.0%
LANOL™ 1688: 12.0%
LANOL™ 14 M: 2.0%
Cetyl alcohol: 0.3%
SCHERCEMOL™ OP: 3%
B Water: q.s.f. 100%
C Polyelectrolyte 2: 0.35%
D SEPICIDE™ CI: 0.2%
SEPICIDE™ HB: 0.5%
Perfume: 0.20%

Procedure

Emulsify B in A at about 75° C.; add C at about 60° C., then D at about 30° C.

### EXAMPLE 37

### Massage Care Gel

Formula
A Polyelectrolyte 1: 3.00%
Water: 30%
B SEPICIDE™ CI: 0.20%
SEPICIDE™ HB: 0.30%
Perfume: 0.05%
C Dye: q.s.
Water: q.s.f. 100%
D MICROPEARL™ SQL: 5.0%
LANOL™ 1688: 2%

Procedure

Prepare A; add B, then C, then D.

### EXAMPLE 38

### Body Milk

Formula
A MONTANOV™ S: 3.0%
Glycerol triheptonate: 10.0%
B Water: q.s.f. 100%
C Polyelectrolyte 1: 1.0%
D Perfume: q.s.
Preservative: q.s.

Procedure

Melt A at about 75° C. Emulsify B in A at 75° C. then add C at about 60° C., then D.

### EXAMPLE 39

### Soothing After-Shave Balsam, Alcohol-Free

Mixture of lauroyl amino acids: 0.1% to 5%
Magnesium aspartate and potassium aspartate: 0.002% to 0.5%
LANOL™ 99: 2%
Sweet almond oil: 0.5%
Water: q.s.f. 100%
Polyelectrolyte 3: 3%
SEPICIDE™ HB: 0.3%
SEPICIDE™ CI: 0.2%
Perfume: 0.4%

### EXAMPLE 40

### Body Milk

MONTANOV™ S: 3.5%
LANOL™ 37T: 8.0%
SOLAGUM™ L: 0.05%
Water: q.s.f. 100%
Benzophenone-3: 2.0%
Dimethicone 350 cPs: 0.05%
Polyelectrolyte 3: 0.8%
Preservative: 0.2%
Perfume: 0.4%

### EXAMPLE 41

### Soothing After-Shave Balsam, Alcohol-Free

LIPACIDE™ PVB: 1.0%
LANOL™ 99: 2.0%
Sweet almond oil: 0.5%
Polyelectrolyte 1: 3.5%
Water: q.s.f. 100%
Perfume: 0.4%
SEPICIDE™ HB: 0.4%
SEPICIDE™ CI: 0.2%

### EXAMPLE 42

### Refreshing After-Shave Gel

LIPACIDE™ PVB: 0.5%
LANOL™ 99: 5.0%
Polyelectrolyte 1: 2.5%
Water: q.s.f. 100%
MICROPEARL™ LM: 0.5%
Perfume: 0.2%
SEPICIDE™ HB: 0.3%
SEPICIDE™ CI: 0.2%

### EXAMPLE 43

### Cream with AHAs

MONTANOV™ 68: 5.0%
LIPACIDE™ PVB: 1.05%
LANOL™ 99: 10.0%
Water: q.s.f. 100%
Gluconic acid: 1.5%
TEA (triethanolamine): 0.9%
Polyelectrolyte 4: 1.5%
Perfume: 0.4%
SEPICIDE™ HB: 0.2%
SEPICIDE™ CI: 0.4%

### EXAMPLE 44

### Gloss Gel

Polyelectrolyte 1: 1.5%
Volatile silicone: 25%
Monopropylene glycol: 25%
Demineralized water: 10%
Glycerol: q.s. 100%

### EXAMPLE 45

### Slimming Gel

Polyelectrolyte 2: 1.5%
Isononyl isononanoate: 2%
Caffeine: 5%
Ethanol: 40%
MICROPEARL™ LM: 2%
Demineralized water: q.s. 100%
Preservative perfume: qs

### EXAMPLE 46

### Milk for Makeup Removal

SIMULSOL™ 165: 4%
MONTANOV™ M 202: 1%
Caprylate-caprate triglyceride: 15%
PECOSIL™ DCT: 1%
Demineralized water: qs
CAPIGEL™ 98: 0.5%
Polyelectrolyte 3: 1%
PROTEOL™ APL: 2%
Sodium hydroxide: q.s. pH=7

### EXAMPLE 47

### “Rinse-Off” Restructuring Cream Mask for Stressed and Brittle Hair

KETROL™T: 0.5%
PECOSIL™ SPP50: 0.75%
N-cocoyl amino acids: 0.70%
Butylene glycol: 3.0%
Polyelectrolyte 4: 3.0%
MONTANOV™ 82: 3.0%
Jojoba oil: 1.0%
LANOL™ P: 6.0%
AMONYL™ DM: 1.0%
LANOL™ 99: 5.0%
SEPICIDE™ HB: 0.3%
SEPICIDE™ CI: 0.2%
Perfume: 0.2%
Water: q.s. 100%

### EXAMPLE 48

### Sunscreen Cream

SIMULSOL™ 165: 3%
MONTANOV™ M 202: 2%
Benzoate C12-C15: 8%
PECOSIL™ PS 100: 2%
Dimethicone: 2%
Cyclomethicone: 5%
Octyl para-methoxy cinnamate: 6%
Benzophenone-3: 4%
Titanium dioxide: 8%
Xanthan gum: 0.2%
Butylene glycol: 5%
Demineralized water: q.s. 100%
Polyelectrolyte 3: 1.5%
Preservative, perfume: qs

### EXAMPLE 49

### Care Gel for Mixed Skin Types

Polyelectrolyte 2: 4%
Vegetable squalane: 5%
Dimethicone: 1.5%
SEPICONTROL™ A5: 4%
Xanthan gum: 0.3%
Water: q.s.: 100%
Preservative, Perfume: qs.

### EXAMPLE 50

### Hair Lotion

Butylene glycol: 3.0%
Polyelectrolyte 2: 3%
SIMULSOL™ 1293: 3.0%
Lactic acid: qs. pH=6
SEPICIDE™ HB: 0.2%
SEPICIDE™CI: 0.3%
Perfume: 0.3%
Water: qs. 100%

### EXAMPLE 51

### Protective and Relaxing Shampoo

Amonyl™ 675 SB: 5.0%
Sodium lauroyl ether sulfate at 28%: 35.0%
Polyelectrolyte 4: 3.0%
SEPICIDE™ HB: 0.5%
SEPICIDE™ CI: 0.3%
Sodium hydroxide: q.s. pH=7.2
Perfume: 0.3%
Dye (FDC blue 1/yellow 5): q.s.
Water: q.s. 100%

### EXAMPLE 52

### “Leave-On” Protectant; Antistress Care for the Hair

KETROL™T: 0.5%
Mixture of cocoyl amino acids: 3.0%
Butylene glycol: 5.0%
DC 1501: 5.0%
Polyelectrolyte 2: 4.0%
SEPICIDE™ HB: 0.5%
SEPICIDE™ CI: 0.3%
Perfume: 0.3%
Water: q.s. 100

### EXAMPLE 53

### Vitamin Cream

SIMULSOL™ 165: 5%
MONTANOV™ 202: 1%
Caprylic/capric triglycerides: 20%
Vitamin A palmitate: 0.2%
Vitamin E acetate: 1%
MICROPEARL™ M 305: 1.5%
Polyelectrolyte 3: 2%
Water q.s. 100%
Preservative, perfume qs

### EXAMPLE 54

### Sunscreen Gel

Polyelectrolyte 4: 3.00%
SEPICIDE™ CI: 0.20%
SEPICIDE™ HB: 0.30%
Perfume: 0.10%
Dye: qs.
Silica: 3.00%
Water: q.s. 100%
Silicone oil: 2.0%
Benzophenone-3: 5.00%

### EXAMPLE 55

### Lip Gloss

Polyelectrolyte 3: 1.50%
Schercemol™ TISC: 15.00%
Vistanol™ NPGC: 15.00%
Candurin Paprika: 0.50%
MONTANOX™ 80: 1.00%
Antaron™ V216: 0.90%
Apricot flavor: 0.20%
SEPICIDE™ HB: 0.50%
C Maltidex™ H16322: q.s. 100%

### EXAMPLE 56

### Terre de Soleil Compacted Powder

Polyelectrolyte 1: 2.00%
Lanol™ 99: 12.00%
SEPIWHITE™ MSH: 1.00%
Talc: 33.00%
MICROPEARL™ M310: 3.00%
Yellow iron oxide: 0.80%
Red iron oxide: 0.30%
Black iron oxide: 0.05%
Mica: qs 100%

### EXAMPLE 57

### Emulsion for Skin Types with Atopic Tendency

ARLACEL™ P135: 2.00%
Polyelectrolyte 2: 1.00%
Lanol™ 1688: 14.00%
Primol™ 352: 8.00%
Glycerol: 5.00%
Water: q.s. 100%
Magnesium sulfate: 0.70%
SEPICIDE™ HB: 0.30%
SEPICIDE™ CI: 0.20%
MICROPEARL™ M310: 5.00%

### EXAMPLE 58

### Soothing Sun Care (Water in Silicone)

Polyelectrolyte 3: 2.00%
DC5225C: 20.00%
DC345: 10.00%
SEPICALM™ VG: 3.00%
Titanium dioxide MT100T: 5.00%
Zinc oxide Z COTE HP1: 5.00%
SEPICIDE™ HB: 0.30%
Perfume: 0.05%
SEPICIDE™ CI: 0.20%
Glycerol: 5.00%
Sodium chloride: 2.00%
Water: q.s. 100%

### EXAMPLE 59

### Multi-Phase Care

Polyelectrolyte 4: 3.00%
C12-15 alkylbenzoate: 25.00%
AQUAXYL™: 3.00%
SEPITONIC™ M3: 1.00%
SEPICIDE™ HB: 0.50%
SEPICIDE™ CI: 0.30%

### EXAMPLE 60

### Self-Tanning Gel

Polyelectrolyte 3: 5.0%
Ethanol: 30%
Dihydroxyacetone: 5%
Menthol: 0.1%
Caffeine: 2.5%
Ivy Extract: 2%
SEPICIDE™ HB: 1%
Water: q.s. 100%

### EXAMPLE 61

### Sunscreen and Self-Tanning Gel

MONTANOV™ S: 3.0%
Glyceryl triheptanoate: 10.0%
LIPACIDE™ PVB: 1.05%
Polyelectrolyte 2: 2.2%
Water: qs. 100%
Dihydroxyacetone: 5%
Perfume: 0.1%
SEPICIDE™ HB: 0.3%
SEPICIDE™ CI: 0.1%
PARSOL™ MCX: 4.0%

### EXAMPLE 62

### Self-Tanning Cream with α-hydroxy Acids

MONTANOV™ 68: 5.0%
LIPACIDE™ PVB: 1.05%
Lanol™99: 10.0%
Water: qs. 100%
Gluconic acid: 1.5%
Dihydroxyacetone: 3%
Triethanolamine: 0.9%
Polyelectrolyte 1: 1.5%
Perfume: 0.4%
SEPICIDE™ HB: 0.2%
SEPICIDE™ CI: 0.4%

### EXAMPLE 63

### Self-Tanning Cream with α-hydroxy Acids for Sensitive Skin

Mixture of N-lauroyl amino acids: 0.1% to 5%
Magnesium aspartate and potassium aspartate: 0.002% to 0.5%
MONTANOV™ 68: 5.0%
Lanol™ 99: 2.0%
Water: qs. 100%
Lactic acid: 1.5%
Dihydroxyacetone: 3.5%
Triethanolamine: 0.9%
Polyelectrolyte 1: 1.5%
Perfume: 0.4%
SEPICIDE™ HB: 0.3%
SEPICIDE™ CI: 0.2%

### EXAMPLE 64

### Self-Tanning Moisturizing Satin Emulsion

SIMULSOL™ 165: 5.0%
Lanol™ 1688: 8.5%
Galam butter: 2%
Liquid paraffin: 6.5%
Lanol™ 14M: 3%
Lanol™ S: 0.6%
Water: 66.2%
Dihydroxyacetone: 3%
Micropearl™ M 100: 5%
Polyelectrolyte 3: 3%
AQUAXYL™: 5%
Vitamin E acetate: 0.20%
Sodium pyrrolidinonecarboxylate: 0.20%
Perfume: 0.2%
SEPICIDE™ HB: 0.5%
SEPICIDE™ CI: 0.3%

The definitions of the commercial products used in the examples are as follows:

SIMULSOL™ 1293 is hydrogenated and ethoxylated castor oil, with an ethoxylation index equal to 40, marketed by the company SEPPIC.

CAPIGEL™ 98 is a liquid thickener based on acrylate copolymer marketed by the company SEPPIC.

KETROL™ T is xanthan gum marketed by the company KELCO.

LANOL™ 99 is isononyl isononanoate marketed by the company SEPPIC.

DC1501 is a mixture of cyclopentasiloxane and dimethiconol marketed by the company DOW CHEMICAL.

MONTANOV™ 82 is an emulsifier based on cetearyl alcohol and cocoylglucoside.

MONTANOV™ 68 (cetearyl glucoside), is a self-emulsifiable composition as described in WO 92/06778, marketed by the company SEPPIC.

MICROPEARL™ M 100 is an ultrafine powder with a very soft feel and a matting effect, marketed by the company MATSUMO.

SEPICIDE™ CI, imidazolidine urea, is a preservative marketed by the company SEPPIC.

PEMULEN™ TR1 is an acrylic polymer marketed by GOODRICH.

SIMULSOL™ 165 is self-emulsifiable glycerol stearate marketed by the company SEPPIC.

LANOL™ 1688 is a non-greasy emollient ester marketed by the company SEPPIC.

LANOL™ 14M and LANOL™ S are consistency factors marketed by the company SEPPIC.

SEPICIDE™ HB, which is a mixture of phenoxyethanol, methylparaben, ethylparaben, propylparaben and butylparaben, is a preservative marketed by the company SEPPIC.

AQUAXYL™ is a moisturizing agent marketed by the company SEPPIC.

SCHERCEMOL™ OP is a non-greasy emollient ester.

LANOL™ P is an additive with stabilizing effect marketed by the company SEPPIC.

PARSOL™ MCX is octyl para-methoxy cinnamate; marketed by the company GIVAUDAN.

MONTANOV™ S is a luster agent, marketed by the company SEPPIC, based on a mixture of alkyl polyglucosides such as those described in WO 95/13863.

MICROPEARL™ SQL is a mixture of microparticles containing squalane, which is released under the action of massaging; it is marketed by the company MATSUMO.

LANOL™ 37T is glycerol triheptanoate, marketed by the company SEPPIC.

SOLAGUM™ L is a carrageenan marketed by the company SEPPIC.

MARCOL™ 82 is a paraffin oil marketed by the company EXXON.

LANOL™ 84D is dioctyl malate marketed by the company SEPPIC.

PARSOL™ NOX is a sun filter marketed by the company GIVAUDAN.

EUSOLEX™ 4360 is a sun filter marketed by the company MERCK.

DOW CORNING™ 245 Fluid is cyclomethicone, marketed by the company DOW CORNING.

LIPACIDE™ PVB is an acylated hydrolyzate of wheat proteins marketed by the company SEPPIC.

MICROPEARL™ LM is a mixture of squalane, polymethylmethacrylate and menthol, marketed by the company SEPPIC.

SEPICONTROL™ AS is a mixture of capryloy glycine, sarcosine, cinnamon extract zylanicum, marketed by the company SEPPIC, such as those described in international patent application PCT/FR98/01313 filed on 23 Jun. 1998.

LANOL™ 2681 is a mixture of copra caprylate and caprate, marketed by the company SEPPIC.

MONTANOV™ 202 is an APG/fatty alcohols composition as described in WO9 98/47610, marketed by the company SEPPIC.

PROTEOL™ APL is a foaming surfactant, marketed by the company SEPPIC.

SCHERCEMOL™ TISC is an ester (tri-isostearyl citrate) marketed by the company SCHER.

VISTANOL™ NPGC is an ester (neopentyl glycol dicaprate) marketed by the company SEWA KASEI.

ANTARON™ V216 is a synthetic polymer (PVP/hexadecene copolymer) distributed by the company UNIVAR.

C MALTIDEX™ H16322 is a polyol (maltitol syrup) marketed by the company CERESTAR.

SEPIWHITE™MSH is a depigmenting agent (N-undecylenoyl phenylalanine) marketed by the company SEPPIC.

DC 345 is a cyclomethicone marketed by the company Dow Corning.

DC 5225C is a mixture of cyclopentasiloxane and dimethicone copolyol marketed by the company DOW CORNING.

SEPICALM™ VG is a soothing agent (sodium palmitoylproline) marketed by the company SEPPIC.

MT100VT is a micronized titanium dioxide that has undergone a surface treatment (aluminum hydroxide/stearic acid) distributed by the company UNIPEX.

Z COTE HP1 is a micronized zinc oxide that has undergone a surface treatment, distributed by GATTEFOSSE.

CANDURIN PAPRIKA is a mixture of potassium and aluminum silicate and iron oxide.

MICROPEARL™ M 310 is an ultrafine powder that has a very soft feel and a matting effect, marketed by the company MATSUMO.

PRIMOL™ 352 is a mineral oil marketed by the company EXXON.

PECOSIL™DCT is Sodium Dimethicone PEG-7 Acetyl Methyltaurate marketed by the company PHOENIX.

PECOSIL™PS 100 is Dimethicone PEG-7 marketed by the company PHOENIX.

1-15. (canceled)

16. A crosslinked anionic polyelectrolyte, resulting from the polymerization of 2-methyl 2-[(1-oxo 2-propenyl)amino] 1-propanesulfonic acid partially salified or completely salified with at least one neutral monomer selected from acrylamide, (2-hydroxy-ethyl) acrylate or N,N-dimethyl acrylamide, and at least one monomer of formula (I): 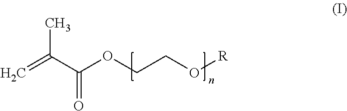
in which R represents a linear or branched alkyl radical having from eight to twenty carbon atoms and n represents a number greater than or equal to one and less than or equal to twenty selected from tetraethoxylated lauryl methacrylate or eicosaethoxylated stearyl methacrylate in the presence of at least one crosslinking agent.

17. The anionic polyelectrolyte as claimed in claim 16, for which the monomer with strong acid function is 2-methyl 2-[(1-oxo 2-propenyl) amino] 1-propanesulfonic acid partially or completely salified in the form of ammonium salt.

18. The anionic polyelectrolyte as claimed in claim 16, for which the neutral monomer is N,N-dimethyl acrylamide.

19. The anionic polyelectrolyte as claimed in claim 18, for which the neutral monomer is acrylamide.

20. The anionic polyelectrolyte as claimed in claim 16, for which the neutral monomer is (2-hydroxy-ethyl) acrylate.

21. The anionic polyelectrolyte as claimed in claim 16, for which in formula (I), R represents an alkyl radical having from 12 to 18 carbon atoms.

22. The anionic polyelectrolyte as claimed in claim 16, selected from the following polymers:
Terpolymer of 2-methyl 2-[(1-oxo 2-propenyl) amino] 1-propanesulfonic acid completely salified in the form of ammonium salt, acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, acrylamide and eicosaethoxylated stearyl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and eicosaethoxylated stearyl methacrylate, crosslinked with trimethylol propanetriacrylate
Terpolymer of 2-methyl 2-[(1-oxo 2-propenyl) amino] 1-propanesulfonic acid completely salified in the form of ammonium, N,N-dimethyl acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, N,N-dimethyl acrylamide and eicosaethoxylated stearyl methacrylate, crosslinked with trimethylol propanetriacrylate.

23. The crosslinked anionic polyelectrolyte, as claimed in claim 16, comprising for 100% of monomers used:
From 20 mol. % to 80 mol. % of monomer units derived from a monomer having a strong acid function;
From 15 mol. % to 75 mol. % of monomer units derived from a neutral monomer;
From 0.5% to 5 mol. % of monomer units derived from a monomer of formula (I) as defined above.

24. The crosslinked anionic polyelectrolyte, as claimed in claim 23, comprising for 100% of monomers used:
From 55 mol. % to 80 mol. % of monomer units derived from a monomer having a strong acid function;
From 15 mol. % to 40 mol. % of monomer units derived from a neutral monomer;
From 1% to 5 mol. % of monomer units derived from a monomer of formula (I) as defined above.

25. A method of at least one of thickening, stabilizing, and emulsifying at least one of a cosmetic, dermopharmaceutical and pharmaceutical topical composition, comprising adding an effective amount of the anionic polyelectrolyte as claimed in claim 16 to said at least one of a cosmetic, dermopharmaceutical and pharmaceutical topical composition.

26. A topical composition, comprising 0.1 and 10 wt. % of the anionic polyelectrolyte as claimed in claim 16.

27. The anionic polyelectrolyte as claimed in claim 17, for which the neutral monomer is N,N-dimethyl acrylamide.

29. The anionic polyelectrolyte as claimed in claim 17, for which in formula (I), R represents an alkyl radical having from 12 to 18 carbon atoms.

30. The anionic polyelectrolyte as claimed in claim 18, for which in formula (I), R represents an alkyl radical having from 12 to 18 carbon atoms.

31. The anionic polyelectrolyte as claimed in claim 19, for which in formula (I), R represents an alkyl radical having from 12 to 18 carbon atoms.

32. The anionic polyelectrolyte as claimed in claim 20, for which in formula (I), R represents an alkyl radical having from 12 to 18 carbon atoms.

33. The anionic polyelectrolyte as claimed in claims 17, selected from the following polymers:
Terpolymer of 2-methyl 2-[(1-oxo 2-propenyl) amino] 1-propanesulfonic acid completely salified in the form of ammonium salt, acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, acrylamide and eicosaethoxylated stearyl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and eicosaethoxylated stearyl methacrylate, crosslinked with trimethylol propanetriacrylate
Terpolymer of 2-methyl 2-[(1-oxo 2-propenyl) amino] 1-propanesulfonic acid completely salified in the form of ammonium, N,N-dimethyl acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, N,N-dimethyl acrylamide and eicosaethoxylated stearyl methacrylate, crosslinked with trimethylol propanetriacrylate.

34. The anionic polyelectrolyte as claimed in claims 18, selected from the following polymers:
Terpolymer of 2-methyl 2-[(1-oxo 2-propenyl) amino] 1-propanesulfonic acid completely salified in the form of ammonium salt, acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, acrylamide and eicosaethoxylated stearyl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and eicosaethoxylated stearyl methacrylate, crosslinked with trimethylol propanetriacrylate
Terpolymer of 2-methyl 2-[(1-oxo 2-propenyl) amino] 1-propanesulfonic acid completely salified in the form of ammonium, N,N-dimethyl acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, N,N-dimethyl acrylamide and eicosaethoxylated stearyl methacrylate, crosslinked with trimethylol propanetriacrylate.

35. The anionic polyelectrolyte as claimed in claims 19, selected from the following polymers:
Terpolymer of 2-methyl 2-[(1-oxo 2-propenyl) amino] 1-propanesulfonic acid completely salified in the form of ammonium salt, acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, acrylamide and eicosaethoxylated stearyl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, 2-hydroxy-ethyl acrylate and eicosaethoxylated stearyl methacrylate, crosslinked with trimethylol propanetriacrylate
Terpolymer of 2-methyl 2-[(1-oxo 2-propenyl) amino] 1-propanesulfonic acid completely salified in the form of ammonium, N,N-dimethyl acrylamide and tetraethoxylated lauryl methacrylate, crosslinked with trimethylol propanetriacrylate;
Terpolymer of 2-methyl 2-[(1-oxo 2-propenyl) amino] 1-propanesulfonic acid completely salified in the form of ammonium salt, N,N-dimethyl acrylamide and eicosaethoxylated stearyl methacrylate, crosslinked with trimethylol propanetriacrylate.
